(19) United States
(12) Patent Application Publication
(10) Pub. No.: US 2004/0126412 A1
(43) Pub. Date: Jul. 1, 2004

(54) Hemorrhoid treatment apparatus

(76) Inventors: Kevin Halloran, Castro Valley, CA (US); Brian K. Ross, Castro Valley, CA (US)

Correspondence Address: BRIAN K. ROSS, Ste. B, 1124 EAST 14th St., San Leandro, CA, 94577, US

(21) Appl. No.: 10/630,415

(22) Filed: Jul. 29, 2003

Related U.S. Application Data
(60) Provisional application No. 60/404,705, filed on Aug. 20, 2002.

(51) Int. Cl. A61K 9/70; A61M 31/00

(52) U.S. Cl. 424/443; 604/500

(57) ABSTRACT

A small, cylindrical-shaped, disposable or reusable, and flexible bladder, cooled or frozen before applied to the anus of the user, and held in place by the user's buttocks. Said bladder is composed of a plastic, rubber or other flexible material sealed at its ends. A cooling agent or compound is contained within the bladder, which is intended to be cooled or frozen in one's refrigerator, icebox or freezer. Upon application, the bladder provides instant relief to the user by cooling the exposed hemorrhoidal tissue and by accelerating the shrinkage of said tissue.

References Cited
U.S. Patent Documents

| | | | |
|---|---|---|---|
| 3809096 | May, 1974 | York | 607/424. |
| 4240436 | Dec., 1980 | Singleton | 607/108. |
| 4563182 | Jan., 1986 | Stoy et al. | 607/113. |
| 4844073 | Jul., 1989 | Pohler | 607/113. |
| 4932397 | Jun., 1990 | McFaul, Sr. | |
| 4938221 | Jul., 1990 | Tuffel | 607/113. |
| 5062425 | Nov., 1991 | Tucker | 604/97. |
| 5192266 | Mar., 1993 | Wilk. | |
| 5263926 | Nov., 1993 | Wilk. | |
| 5707645 | Jan., 1998 | Wierson | |

### DESCRIPTION

The unit is approximately 3½ inches long and is cylindrical in shape. The unit is approximately ½ to ⅝ inches in diameter and is to be made out of a flexible rubber or plastic material. The unit is filled with a cooling agent or compound for prolonged cooling, enabling shrinkage of the hemorrhoidal tissues. The unit provides prompt cooling of the affected area providing temporary relief from the discomfort caused by irritated hemorrhoidal tissues within minutes.

### BRIEF DESCRIPTION OF THE FIGURES/DRAWINGS

FIG. 1 is a perspective view of the unit from its side, depicting the units cylindrical shape and its length, which can range from 2 & ½ inches to 3 & ½ inches.

FIG. 1A is a perspective view of the unit from its side, depicting the unit in its alternative, rectangular or cube shape and its length, which can range from 2 & ½ inches to 3 & ½ inches.

FIG. 1B is an inset depicting the placement of the cooling agent or compound within the bladder of the unit.

FIG. 2 is a perspective view of the unit from its end, depicting its diameter (½-⅝″) in its cylindrical form.

FIG. 2A is another perspective view of the unit from its end, once again depicting its diameter (½-⅝″), but depicting the unit it its alternative, cube form.

FIG. 3 is a perspective view of the unit from its top depicting the units rectangular or cylindrical shape and its length, which can range from 2 & ½ inches to 3 & ½ inches.

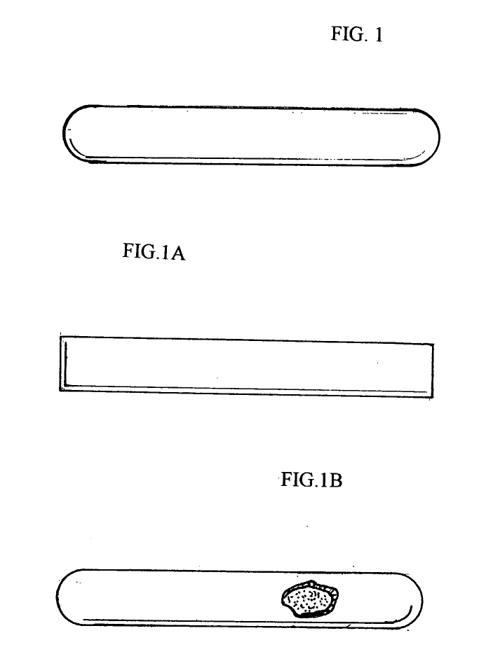

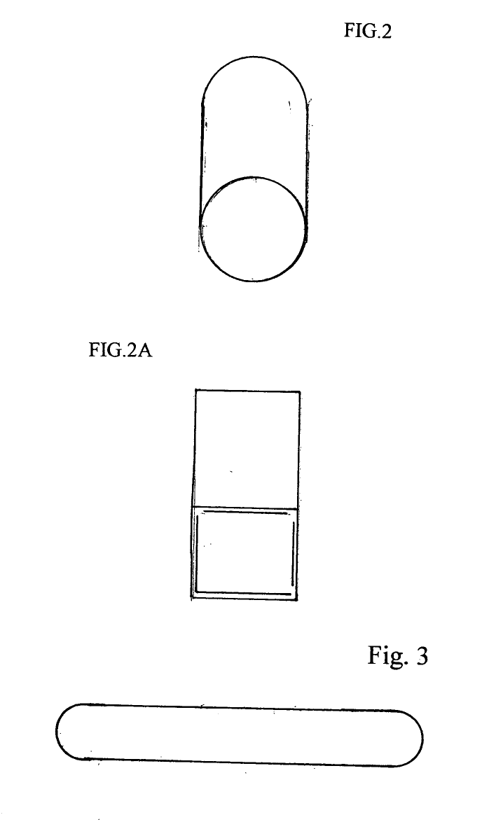

What is claimed is:

1. A reusable or disposable cold bladder having a preformed cylindrical shape for treatment of the anal region afflicted by hemorrhoids compromising:
A 3½ inch long, rectangular or cylindrical shaped unit, approximately ½ to ⅝ inches in diameter, whereby (a) the user applies the unit between the user's buttocks atop the exposed hemorrhoidal tissues until the maximum comfort is achieved, and (b) the unit is then removed and sterilized or otherwise cleaned for future applications or manufactured in a disposable form.

2. The invention of claim 1 wherein said cold bladder comprises a cooling agent or compound intended to provide prolonged cooling, enabling shrinkage of the hemorrhoidal tissues.

3. The invention of claim 1 wherein said cold bladder is composed of a flexible, plastic or rubber material, such as polyethylene.
